(19) United States
(12) Patent Application Publication
(10) Pub. No.: US 2021/0379306 A1
(43) Pub. Date: Dec. 9, 2021

(54) VENTILATION THERAPY APPARATUS AND CONTROL METHOD

(71) Applicant: BMC MEDICAL CO., LTD., Beijing (CN)

(72) Inventors: Qingsong WANG, Beijing (CN); Zhi ZHUANG, Beijing (CN)

(21) Appl. No.: 17/288,040

(22) PCT Filed: Oct. 24, 2019
(86) PCT No.: PCT/CN2019/113126

§ 371 Date: Apr. 23, 2021

(30) Foreign Application Priority Data
Oct. 26, 2018 (CN) 201811261603.1

(51) Int. Cl. A61M 16/00; A61M 16/16; A61M 16/10; A61M 16/06; A61M 16/08

(52) U.S. Cl. CPC A61M 16/022; A61M 16/16; A61M 16/1075; A61M 2016/003; A61M 16/0875; A61M 2205/3368; A61M 2205/3331; A61M 16/0666

(57) ABSTRACT

A ventilation therapy apparatus and a control method, includes: an apparatus body, a respiratory pipe and a patient interface. The apparatus body further includes: a signal acquisition module, a target pressure acquisition module and a first control module. The signal acquisition module is configured for acquiring an output pressure value and an output flow value of a signal collection point of the apparatus body; the target pressure acquisition module is configured for acquiring a target pressure value at the patient interface; the first calculation module is configured for, calculating an actual pressure value at the patient interface according to the output pressure value and the output flow value of the signal collection point; the first control module is configured for adjusting an output flow of the apparatus body according to the actual pressure value and the target pressure value. In the present disclosure, it is capable to determine the actual pressure value at the patient interface by the output parameters feedback of the signal collection point of the apparatus body, and determine the patient's respiratory state according to the comparison between the actual pressure value and the target pressure value, and output the gas with a corresponding threshold, therefore the gas pressure of the airflow received by the patient may reach a preset target pressure range, and achieve the therapeutic effect.

### CROSS REFERENCE TO RELEVANT APPLICATIONS

The present disclosure claims the priority of the Chinese patent application filed by State Intellectual Property Office of The P.R.C on Oct. 26, 2018 with the application number of 201811261603.1, and the title of “VENTILATION THERAPY APPARATUS AND CONTROL METHOD”, the entire contents of which are incorporated herein by reference.

### TECHNICAL FIELD

The present disclosure relates to the medical equipment field and, more particularly, to a ventilation therapy apparatus and a control method.

### BACKGROUND

In modern clinical medicine, ventilation therapy apparatuses play a very important role in the field of modern medicine. Ventilation therapy apparatus is a vital medical apparatus that can prevent and treat respiratory failure, reduce complications, and save and prolong the lives of patients, which may mix pure oxygen with air and provide to the patients.

The therapy mode of the conventional ventilation therapy apparatus is to form an enclosed gas path between the patient and the ventilation therapy apparatus, namely to form a sealed state with the patient's facial area through a patient interface (usually a ventilation mask), the gas exhaled by the patient can be discharged by a specific gas path or a specially designed vent, therefore when the ventilation mask and a respiratory pipe are well worn, an exhaust channel of the patient is fixed. The ventilation therapy apparatus with this therapy mode may directly control an output pressure of the ventilation therapy apparatus through monitoring a pressure at the patient, and keep the pressure at the patient end to be equal to an expected output pressure value.

However, if the ventilation therapy apparatus constructs a semi-open gas path, that is, the patient interface and the patient are not sealed, the gas outputted by the ventilation therapy apparatus may directly leak through a gap between the patient interface and the patient's nasal cavity. At this time, the ventilation therapy apparatus is unable to directly monitor or obtain the pressure at the end of the gas path, and therefore it cannot control the pressure at the end of the gas path.

### SUMMARY

The present disclosure provides a ventilation therapy apparatus and a control method, to solve the problem that in the semi-open gas path, the ventilation therapy apparatus in the prior art cannot monitor the pressure at the end of the gas path, and thus cannot achieve the control of the pressure at the end of the gas path.

In order to solve the above technical problem, the present disclosure is realized as follows:

In the first aspect, a ventilation therapy apparatus is provided, comprising: an apparatus body, configured for outputting gas with a preset pressure and a preset flow, wherein the apparatus body includes a gas outlet;

a respiratory pipe, comprising a first end and a second end which communicates with each other, and the first end of the respiratory pipe communicates with the gas outlet;

a patient interface, wherein the second end of the respiratory pipe is connected to the patient interface, the patient interface is configured for being worn on a patient's nasal cavity, when the patient interface is worn on the patient's nasal cavity, a gas outlet gap is disposed between the patient interface and the patient's nasal cavity;

wherein the apparatus body further comprises:

a signal acquisition module, configured for acquiring an output pressure value and an output flow value of a signal collection point of the apparatus body;

a target pressure acquisition module, configured for acquiring a target pressure value at the patient interface;

a first calculation module configured for, calculating an actual pressure value at the patient interface according to the output pressure value and the output flow value of the signal collection point;

a first control module configured for, adjusting an output flow of the apparatus body according to the actual pressure value and the target pressure value;

when the actual pressure value is larger than the target pressure value, reducing the output flow of the apparatus body; and

when the actual pressure value is less than the target pressure value, rising the output flow of the apparatus body.

In the embodiment of the present disclosure, it is capable to determine the actual pressure value at the patient interface by the output pressure value and the output flow value of the signal collection point of the apparatus body, and adjust the output flow of the apparatus body according to comparison between the actual pressure value and the target pressure value, therefore the gas pressure of the airflow received by the patient may reach a preset target pressure range, and achieve the therapeutic effect.

Optionally, the first calculation module comprises:

a gas resistance pressure acquisition module, configured for acquiring a gas resistance pressure value from the signal collection point to the patient interface; and

a second calculation module, configured for subtracting the gas resistance pressure value from the output pressure value, and obtaining the actual pressure value.

In the embodiment of the present disclosure, it may obtain accurate actual pressure value by acquiring the gas resistance pressure value from the signal collection point to the patient interface, and subtracting the gas resistance pressure value from the output pressure value.

Optionally, the gas resistance pressure acquisition module comprises:

a gas resistance characteristic acquisition module, configured for, acquiring, under different pressure states, corresponding test flow values through a flow acquisition module when the patient interface is vacant, and acquiring a gas resistance characteristic from the signal collection point to the patient interface, wherein the gas resistance characteristic includes a correspondence relationship between the output pressure value and the output flow value;

a gas resistance pressure acquisition unit configured for, acquiring the corresponding gas resistance pressure value, according to the output flow value of the apparatus body in working state and the corresponding gas resistance characteristic;

wherein the second calculation module is further configured for, subtracting the corresponding gas resistance pressure value from the output pressure value of the apparatus body, and obtaining the actual pressure value.

In the embodiment of the present disclosure, the flow acquisition module may accurately acquire, under different pressure states, the correspondence relationship between the output pressure value and the output flow value from the signal collection point to the patient interface when the patient interface is vacant. The gas resistance pressure value obtained thereby is more accurate, which in turn makes the actual pressure value more accurate.

Optionally, the signal collection point is disposed at the gas outlet of the apparatus body.

In the embodiment of the present disclosure, the flow acquisition module may acquire the gas resistance characteristic from the signal collection point to the patient interface, the signal collection point is disposed at the gas outlet of the apparatus body, which may more accurately obtain an actual input pressure or flow at the first end of the respiratory pipe.

Optionally, the target pressure value comprises a target pressure value of an inspiratory phase and a target pressure value of an exhalation phase;

the ventilation therapy apparatus includes a determination module, the determination module is configured for determining a respiratory phase according to the output pressure value and the output flow value acquired by the signal acquisition module, the respiratory phase includes the inspiratory phase and the exhalation phase;

when the determination module determines that the current is the inspiratory phase, the first control module adjusts the output flow of the apparatus body according to the actual pressure value and the target pressure value of the inspiratory phase; and

when the determination module determines that the current is the exhalation phase, the first control module adjusts the output flow of the apparatus body according to the actual pressure value and the target pressure value of the exhalation phase.

In the embodiment of the present disclosure, when the patient is inhaling, the total flow outputted by the ventilation therapy apparatus is larger than the total flow by the ventilation therapy apparatus when the patient is exhaling. If the actual pressure value is less than the target pressure value, then it is determined that the patient is inhaling. Adjusting the output flow of the apparatus body according to the respiratory requirements corresponding to the inspiratory phase and the exhalation phase in the respiratory phase, respectively, therefore it may be supplied on demand, and avoid waste.

Optionally, the apparatus body further comprises a positive pressure gas source and a humidifier, the positive pressure gas source is configured for providing an output gas, and the humidifier is configured for heating and humidifying the output gas, wherein the humidifier is connected to an output end of the positive pressure gas source.

In the embodiment of the present application, the humidifier is used to heat and humidify the gas provided by the positive pressure gas source, therefore it may meet the respiratory requirements of the user and improve the respiratory effect.

Optionally, the positive pressure gas source comprises a gas source body capable of outputting gas with a preset flow, and/or a centrifugal fan configured for pressurize air, wherein the maximum rotation speed of the centrifugal fan is larger than or equal to 20000 r/min.

In the embodiment of the present disclosure, the positive pressure gas source includes the gas source body capable of outputting gas with the preset flow, and/or the centrifugal fan configured for pressurize air, the method of obtaining the positive pressure gas source is simply and convenient.

Optionally, the respiratory pipe further comprises a heating element configured for heating gas passing through the respiratory pipe, the rated power of the heating element is larger than 20 watts.

In the embodiment of the present disclosure, the heating element configured for heating gas passing through the respiratory pipe is disposed in the respiratory pipe, which may heat the temperature of the output gas in a cold environment, and improve the respiratory experience of the patient.

Optionally, the respiratory pipe further comprises a temperature sensor, configured for monitoring the temperature of the gas passing through the respiratory pipe.

In the embodiment of the present disclosure, the temperature sensor may monitor the temperature of the gas passing through the respiratory pipe in real time, therefore the ventilation therapy apparatus according to the monitored temperature, carries out the operation of correspondingly controlling the heating element to heat gas, and stops heating at the same time when the temperature is too high, which may improve the respiratory experience of the patient.

Optionally, the respiratory pipe and the apparatus body are connected through a gas path and a circuit, and the circuit and the gas path are on and off simultaneously.

In the embodiment of the present disclosure, the respiratory pipe and the apparatus body are connected through the gas path, which may output the gas provided by the apparatus body to the patient. In addition, the respiratory pipe and the apparatus body are connected through the circuit, and the electrical device in the respiratory pipe may also be electrically connected to the apparatus body, to realize the corresponding functions of the electrical device.

Optionally, the ventilation therapy apparatus comprises a second control module;

when the patient interface is worn on the patient's nasal cavity, the second control module is configured for adjusting the output pressure value of the apparatus body to the target pressure value; and

when the patient interface is not worn on the patient's nasal cavity, the second control module is configured for adjusting the output pressure value of the apparatus body to a preset pressure value, or the second control module is configured for controlling the apparatus body to stop running.

In the embodiment of the present disclosure, when the patient interface is worn on the patient's nasal cavity, the second control module is configured for automatically adjusting the output pressure value of the apparatus body to the target pressure value, therefore the patient may accept oxygen supply quickly. When the patient did not use an oxygen supply system of the ventilation therapy apparatus, the patient interface is exposed to the air, meanwhile, the ventilation therapy apparatus may continue to output a smaller output flow, to ensure consistent temperature and humidity inside the respiratory pipe, or directly control the apparatus body to stop running, which may save power.

In the second aspect, a method for controlling the ventilation therapy apparatus is provided, the ventilation therapy apparatus constructs a semi-open gas path, the method comprises:

acquiring an output pressure value and an output flow value of a signal collection point of an apparatus body;

acquiring a target pressure value at a patient interface;

calculating an actual pressure value at the patient interface according to the output pressure value and the output flow value of the signal collection point;

adjusting an output flow of the apparatus body according to the actual pressure value and the target pressure value;

when the actual pressure value is larger than the target pressure value, reducing the output flow of the apparatus body; and

when the actual pressure value is less than the target pressure value, rising the output flow of the apparatus body.

In the embodiment of the present disclosure, it is capable to determine the actual pressure value at the patient interface by the output pressure value and the output flow value of the signal collection point of the apparatus body, and adjust the output flow of the apparatus body according to comparison between the actual pressure value and the target pressure value, therefore the gas pressure of the airflow received by the patient may reach a preset target pressure range, and achieve the therapeutic effect.

Optionally, the step of calculating an actual pressure value at the patient interface according to the output pressure value and the output flow value of the signal collection point, comprises:

acquiring a gas resistance pressure value from the signal collection point to the patient interface; and

subtracting the gas resistance pressure value from the output pressure value, and obtaining the actual pressure value.

In the embodiment of the present disclosure, it may obtain accurate actual pressure value by acquiring the gas resistance pressure value from the signal collection point to the patient interface, and subtracting the gas resistance pressure value from the output pressure value.

Optionally, the step of calculating an actual pressure value at the patient interface according to the output pressure value and the output flow value of the signal collection point, comprises:

acquiring the output pressure value and the output flow value of the signal collection point of an apparatus body;

acquiring, under different pressure states, corresponding test flow values when the patient interface is vacant, and acquiring a gas resistance characteristic from the signal collection point to the patient interface, wherein the gas resistance characteristic includes a correspondence relationship between the output pressure value and the output flow value;

acquiring the corresponding gas resistance pressure value according to the output flow value of the apparatus body in working state and the corresponding gas resistance characteristic; and

subtracting the corresponding gas resistance pressure value from the output pressure value of the apparatus body, and obtaining the actual pressure value.

In the embodiment of the present disclosure, the flow acquisition module may accurately acquire, under different pressure states, the correspondence relationship between the output pressure value and the output flow value from the signal collection point to the patient interface when the patient interface is vacant. The gas resistance pressure value obtained thereby is more accurate, which in turn makes the actual pressure value more accurate.

Optionally, the method further comprises:

when the patient interface is worn on the patient's nasal cavity, adjusting the output pressure value of the apparatus body to the target pressure value; and

when the patient interface is not worn on the patient's nasal cavity, adjusting the output pressure value of the apparatus body to a preset pressure value which is less than the target pressure value, or controlling the apparatus body to stop running.

In the embodiment of the present disclosure, when the patient interface is worn on the patient's nasal cavity, the second control module is configured for automatically adjusting the output pressure value of the apparatus body to the target pressure value, therefore the patient may accept oxygen supply quickly. When the patient did not use an oxygen supply system of the ventilation therapy apparatus, the patient interface is exposed to the air, meanwhile, the ventilation therapy apparatus may continue to output a smaller output flow, to ensure consistent temperature and humidity inside the respiratory pipe, or directly control the apparatus body to stop running, which may save power.

Optionally, after acquiring an output pressure value and an output flow value of a signal collection point of an apparatus body, the method further comprises:

determining a respiratory phase according to the output pressure value and the output flow value, wherein the respiratory phase includes the inspiratory phase and the exhalation phase;

acquiring a target pressure value of the inspiratory phase and a target pressure value of the exhalation phase at the patient interface;

if it is determined that the he current is inspiratory phase, adjusting the output flow of the apparatus body according to the actual pressure value and the target pressure value of the inspiratory phase; and

if it is determined that the he current is exhalation phase, adjusting the output flow of the apparatus body according to the actual pressure value and the target pressure value of the exhalation phase.

In the embodiment of the present disclosure, when the patient is inhaling, the total flow outputted by the ventilation therapy apparatus is larger than the total flow by the ventilation therapy apparatus when the patient is exhaling. If the actual pressure value is less than the target pressure value, then it is determined that the patient is inhaling. According to the respiratory requirements respectively corresponding to the inspiratory phase and the exhalation phase in the respiratory phase, adjusting the output flow of the apparatus body respectively, therefore it may be supplied on demand, and avoid waste.

In the third aspect, a computer program is provided, comprises a computer readable code, when the computer readable code is run on a computing processing device, causing the computing processing device to execute the method for controlling the ventilation therapy apparatus anyone of the above ventilation therapy apparatus.

In the fourth aspect, a computer readable medium is provided, storing the above computer program.

The ventilation therapy apparatus and the control method provided in the embodiment of the present disclosure, includes an apparatus body, a respiratory pipe and a patient interface. The apparatus body further includes a signal acquisition module, a target pressure acquisition module and a first control module. The signal acquisition module is configured for acquiring the output pressure value and the output flow value of the signal collection point of the apparatus body; the target pressure acquisition module is configured for acquiring the target pressure value at the patient interface; the first calculation module is configured for calculating an actual pressure value at the patient interface according to the output pressure value and the output flow value of the signal collection point; the first control module is configured for adjusting the output flow of the apparatus body according to the actual pressure value and the target pressure value; when the actual pressure value is larger than the target pressure value, the first control module may reduce the output flow of the apparatus body; and when the actual pressure value is less than the target pressure value, the first control module may rise the output flow of the apparatus body. In the present disclosure, it is capable to determine the actual pressure value at the patient interface by the output pressure value and the output flow value of the signal collection point of the apparatus body, and adjust the output flow of the apparatus body according to comparison between the actual pressure value and the target pressure value, therefore the gas pressure of the airflow received by the patient may reach a preset target pressure range, and achieve the therapeutic effect.

The above description is only an overview of the technical solution of the present disclosure, in order to understand the technical means of the present disclosure more clearly, it may be implemented in accordance with the content of the specification, and in order to make the above and other purposes, features and advantages of the present disclosure more obvious and understandable, the following specifically cites the specific implementation of the present disclosure.

### BRIEF DESCRIPTION OF THE DRAWINGS

In order to more clearly explain the technical solutions in the embodiments of the present disclosure or the prior art, the following will briefly introduce the drawings that need to be used in the description of the embodiments or the prior art. Obviously, the drawings in the following description are some embodiments of the present disclosure. For those of ordinary skill in the art, other drawings may be obtained based on these drawings without creative work.

FIG. 1 is a structural block diagram of the ventilation therapy apparatus according to an embodiment of the present disclosure;

FIG. 2 is a schematic diagram showing a flow-time of the patient's respiratory process according to an embodiment of the present disclosure;

FIG. 3 is a structural block diagram of the first calculation module according to an embodiment of the present disclosure;

FIG. 4 is a schematic diagram showing a flow-pressure drop of the patient's respiratory process according to an embodiment of the present disclosure;

FIG. 5 is a flow chart showing steps of the method for controlling the ventilation therapy apparatus according to an embodiment of the present disclosure;

FIG. 6 is a computing processing device that may implement the method according to the present disclosure provided by an embodiment of the present disclosure; and

FIG. 7 is a portable or fixed storage module according to an embodiment of the present disclosure.

### DETAILED DESCRIPTION

In order to make the purpose, technical solutions and advantages of the embodiments of the present disclosure clearer, the technical solutions in the embodiments of the present disclosure will be described clearly and completely in conjunction with the accompanying drawings in the embodiments of the present disclosure. Obviously, the described embodiments is a part of the embodiments of the present disclosure, but not all of the embodiments. Based on the embodiments in the present disclosure, all other embodiments obtained by those of ordinary skill in the art without creative work shall fall within the protection scope of the present disclosure.

The following describes in detail the ventilation therapy apparatus and the control method according to the present disclosure by listing several specific embodiments.

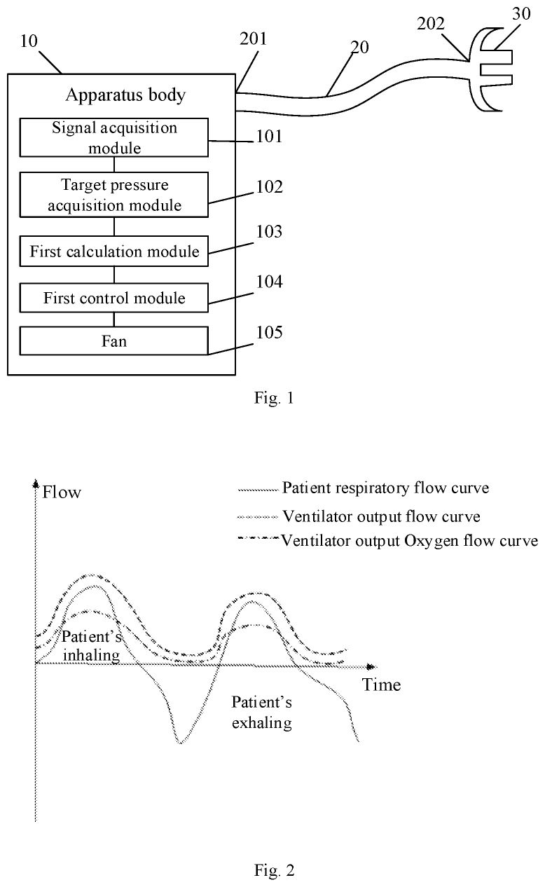

Referring to FIG. 1, there is shown a structural block diagram of the ventilation therapy apparatus according to an embodiment of the present disclosure, comprising an apparatus body 10, configured for outputting gas with a preset pressure and a preset flow, and the apparatus body 10 includes a gas outlet; a respiratory pipe 20, the respiratory pipe 20 including a first end 201 and a second end 202 which communicates with each other, wherein the first end 201 of the respiratory pipe 20 communicates with the gas outlet; a patient interface 30, wherein the second end 202 of the respiratory pipe 20 is connected to the patient interface 30, the patient interface 30 is configured for being worn on a patient's nasal cavity, when the patient interface 30 is worn on the patient's nasal cavity, a gas outlet gap is disposed between the patient interface 30 and the patient's nasal cavity.

Wherein, the ventilation therapy apparatus may be applied in an open gas path, and also may be applied in a semi-open gas path. The semi-open gas path thereof includes a situation that the patient interface 30 is worn on the patient's nasal cavity, a vacant state of the patient interface 30 resulting by that the patient interface 30 is not worn on the patient's nasal cavity, which is a complete open gas path. In the embodiment of the present disclosure, it may adjust correspondingly the output flow of the apparatus body 10 by the comparison result of an actual pressure value and a target pressure value at the patient interface 30.

In addition, Referring to FIG. 1, the structural of the patient interface 30 includes two branch tubes divided at the end, which deliver gas to the two nostrils of the patient separately. An inner diameter of the branch tube at the end of the patient interface 30 is larger than 4 mm, a length is larger than 4 mm, and a thinnest part of a tube wall is less than 0.5 mm. Because these two branch tubes are not an oxygen suction tubes, the flow rate of the oxygen suction tube is usually only 5 to 15 liters per minute, however, these two branch tubes need a large enough airflow (more than 60 liters per minute (LPM)) to generate the required positive pressure, therefore their output flow is relatively large, so the inner diameter is larger than an inner diameter of the conventional oxygen suction tube. But if an outer diameter of the branch tube at the end is also thick, it will hinder the nostril exhalation, and contact with an inner wall will make the patient feel uncomfortable. Therefore, the tube wall should be as thin as possible and not take up an exhaust area of the nostril.

Wherein, the apparatus body 10 further comprises: a signal acquisition module 101, configured for acquiring an output pressure value and an output flow value of a signal collection point of the apparatus body 10.

Specifically, the second end 202 of the respiratory pipe 20 is configured for outputting the gas provided by the apparatus body 10, and the pressure and the flow of the gas outputted may be controlled. A fan 105 may also be disposed inside the apparatus body 10, the fan 105 may be driven by a motor to rotate at a corresponding preset rotating speed, therefore the external air is sucked in the apparatus body 10, and the airflow with different pressure and flow thresholds is outputted through the respiratory pipe 20.

Preferably, the signal acquisition module 101 is configured for acquiring the output pressure value and the output flow value of the signal collection point of the apparatus body 10. Wherein, the signal collection point of the apparatus body 10 may be the gas outlet of the apparatus body 10 or a position close to the gas outlet, and preferably, is configured to obtain the actual input pressure P₁ or flow rate F₀ of the first end 201 of the respiratory pipe 20.

A target pressure acquisition module 102, acquiring a target pressure value at the patient interface 30. The target pressure value may be a pressure value inputted directly into the ventilation therapy apparatus according to the patient's respiratory state, may also be a pressure value suitable for the patient's state automatically learned by the ventilation therapy device based on the patient's respiratory state for a period of time. The target pressure value may be a specific value or a threshold value suitable for the patient's state.

It should be noted that the target pressure value is larger than 0, that is, the target pressure value is larger than the atmospheric pressure value. The target pressure value is larger than the atmospheric pressure value because the ventilation therapy apparatus is applied in the open gas path or the semi-open gas path, and positive pressure must be maintained in the nasal cavity to ensure that the human body does not directly inhale external air.

In practical applications, referring to FIG. 2, there is shown a schematic diagram showing a flow-time of the patient's respiratory process according to the present disclosure. When the patient uses the ventilation therapy apparatus to breathe, the total flow of the inhaled gas will change with time. Therefore, for a conventional ventilation therapy apparatus, in order to optimize the patient's respiratory experience, the patient may be provided with two different levels positive pressure during the patient's exhalation and inhalation. For a high-flow oxygen therapy apparatus, it may provide a larger flow when the patient inhales, to facilitate the patient inhale more gas, and provide a smaller flow when exhaling, to avoid blockage of the patient's airway. At the same time, for the high-flow oxygen therapy apparatus, oxygen supply is introduced. Therefore, the high-flow oxygen therapy apparatus may also ensure a constant oxygen concentration in the outputted gas to ensure a stable therapeutic effect.

In the embodiment of the present disclosure, before the patient uses the ventilation therapy apparatus, a target pressure value Pₜ will be preset according to their own conditions, and the target pressure value Pₜ is the pressure at the patient interface in an ideal state. According to the airflow pressure P₁ and the airflow flow F₀ outputted by the apparatus body 10, the actual pressure P₂ at the patient interface may be calculated. Through real-time monitoring of P₂ and comparing P₂ with the target pressure value Pₜ, it may be determined which preset state the apparatus body is in, that is the exhalation state or the inhalation state, and according to the current state of the apparatus body, the fan of the apparatus body 10 is controlled to run at the corresponding preset rotating speed, and outputs the airflow corresponding to the preset threshold, for example, when it is determined that the patient is inhaling, the air supply system of the ventilation therapy apparatus outputs a flow that is slightly larger than the patient's inhalation volume for auxiliary inhalation; when the patient is determined to exhale, the air supply system of the ventilation therapy apparatus outputs a smaller flow rate, to prevent the patient's exhaled gas from flowing back to the ventilation therapy apparatus.

It should be noted that the target pressure value Pₜ needs to be a positive pressure value, that is, the target pressure value Pₜ is larger than the atmospheric pressure value. Because in the embodiment of the present disclosure, the ventilation therapy apparatus uses an open gas path, it is necessary to ensure that the positive pressure is maintained in the nasal cavity, to ensure that the human body will not directly inhale outside air.

A first calculation module 103, calculating an actual pressure value at the patient interface according to the output pressure value and the output flow value of the signal collection point.

In the embodiment of the present disclosure, the air flow characteristics in the respiratory pipe 20 are certain, according to an energy equation of a fluid, a steady flow of the incompressible fluid in the tube has the following formula:

$$\frac{{U}^{2}}{2} + \frac{p}{ρ} + e + Π = const$$

Wherein, U is a flow rate of the fluid, p is a pressure of the fluid, ρ is a density of the fluid, e is an internal energy of the fluid, Π is a potential energy, and const is a constant, which means that in a fluid system, such as airflow and water flow, the faster the flow rate, the less the pressure generated by the fluid.

In addition, an air flow resistance has the following formula:

F=½CρSU²

Wherein, F is an air resistance, p is the density of the fluid, C is a resistance coefficient, S is a windward area.

Therefore, combining the inferences derived from the above two formulas, after an incompressible fluid flows through a tube with a length of L and a lateral area of S at a certain flow rate, the pressure changes as follows:

ΔP=P₁−P₂=ρ(e₂−e₁)=ρ×F×L=½Cρ²SU²L∝U²

That is, the pressure drop of the incompressible fluid (the pressure drop is a value of the pressure P₁ at the first end 201 of the respiratory pipe 20 minus the pressure at the patient interface) and the flow rate are quadratic. However, the gas is a compressible fluid, so when the pressure drops, the density ρ will increase slightly.

Therefore, either the theoretical output flow rate Fₜ may be calculated from P₁, or the pressure drop ΔP of the air flow through the tube may be calculated from F₀, and according to the formula P₁=ΔP+P₂, the value of the airflow pressure P₂ at the patient interface may be obtained.

A first control module 104, adjusting an output flow of the apparatus body 10 according to the actual pressure value and the target pressure value; when the actual pressure value is larger than the target pressure value, reducing the output flow of the apparatus body 10; and when the actual pressure value is less than the target pressure value, rising the output flow of the apparatus body 10.

Specifically, in the embodiment of the present disclosure, before the patient uses the air supply system of the ventilation therapy apparatus, the target pressure value Pₜ will be preset according to their own conditions, and the target pressure value Pₜ is the pressure value in an ideal state free from the interference of the pressure drop. When the patient uses the ventilation therapy apparatus, due to the interference of the pressure drop, which will cause the actual pressure P₂ at the patient interface is different from the target pressure value Pₜ, and the influence of this pressure drop on the actual pressure P₂ at the patient interface may be determined by the pressure compensation module 103 based on the comparison result between P₂ and Pₜ. If the first control module 104 determines that the actual pressure P₂ at the patient interface is larger than the target pressure value Pₜ, it is determined that the current working state of the apparatus body 10 is the exhalation state, meanwhile a control instruction is sent to the motor by the first control module 104, therefore the motor drives the fan 105 to work at a lower rotating speed, and output a smaller first output threshold airflow, to prevent the patient's exhaled gas from flowing back to the ventilation therapy apparatus, until the actual pressure value is equal to the target pressure value.

It should be noted that, the first control module 104 may also be a compensation module independent of the apparatus body 10, to output a compensation airflow to the respiratory pipe 20. For example, the first control module 104 may be a compensation fan or a compensation gas cylinder disposed independently of the fan 105, when the actual pressure value P₂ is larger than the target pressure value Pₜ, outputs the corresponding compensation air flow, therefore the apparatus body 10 outputs the air flow at a smaller first output threshold; when the actual pressure value P₂ is less than the target pressure value Pₜ, outputs the corresponding compensation air flow, therefore the apparatus body 10 outputs the air flow at a larger second output threshold.

Specifically, if the first control module 104 determines that the actual pressure P₂ at the patient interface is less than the target pressure value Pₜ, then it is determined that the current working state of the apparatus body 10 is the inhalation state. At this time, a control instruction is sent to the motor by the first control module 104, therefore the motor drives the fan 105 to work at a relatively high rotating speed, and output a larger second output threshold airflow, to perform auxiliary inhalation.

In summary, a ventilation therapy apparatus according to the embodiment of the present disclosure, includes: an apparatus body, a respiratory pipe and a patient interface. The apparatus body further includes: a signal acquisition module, a target pressure acquisition module and a first control module. The signal acquisition module is configured for acquiring the output pressure value and the output flow value of the signal collection point of the apparatus body; the target pressure acquisition module is configured for acquiring the target pressure value at the patient interface; the first calculation module is configured for, calculating the actual pressure value at the patient interface according to the output pressure value and the output flow value of the signal collection point; the first control module is configured for adjusting an output flow of the apparatus body according to the actual pressure value and the target pressure value. In the present disclosure, it is capable to determine the actual pressure value at the patient interface by the output parameters feedback of the signal collection point of the apparatus body, determine the patient's respiratory state according to the comparison between the actual pressure value and the target pressure value, and output the gas with a corresponding threshold, therefore the gas pressure of the airflow received by the patient may reach a preset target pressure range, and achieve the therapeutic effect.

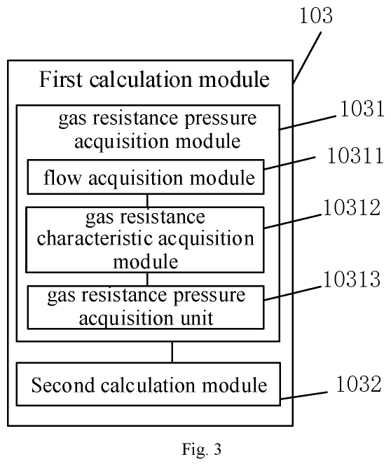

Optionally, referring to FIG. 3, it is shown a structural block diagram of the first calculation module according to an embodiment of the present disclosure, the first calculation module 103 comprises:

a gas resistance pressure acquisition module 1031, configured for acquiring a gas resistance pressure value from the signal collection point to the patient interface. The gas resistance pressure acquisition module 1031 is configured for acquiring the pressure value and the flow value of the signal collection point in the apparatus body 10, wherein the signal collection point in the apparatus body 10 may be the gas outlet of the apparatus body 10 or a position close to the gas outlet, and preferably, is configured to obtain the actual input pressure P₁ or flow rate F₀ of the first end 201 of the respiratory pipe 20.

A second calculation module 1032, configured for subtracting the gas resistance pressure value from the output pressure value, and obtaining the actual pressure value. Specifically, according to the above formula P₁=ΔP+P₂, the value of the airflow pressure P2 at the patient interface may be obtained as P₁−ΔP.

Optionally, referring to FIG. 3, the gas resistance pressure acquisition module 1031 comprises:

a flow acquisition module 10311 configured for, acquiring the output pressure value and the output flow value of the signal collection point of the apparatus body;

A gas resistance characteristic acquisition module 10312 configured for, acquiring, under different pressure states, corresponding test flow values through the flow acquisition module when the patient interface is vacant, and acquiring a gas resistance characteristic from the signal collection point to the patient interface, the gas resistance characteristic includes a correspondence relationship between the output pressure value and the output flow value.

A gas resistance pressure acquisition unit 10313 configured for, according to the output flow value of the apparatus body in working state and the corresponding gas resistance characteristic, acquiring the corresponding gas resistance pressure value.

The second calculation module 1032 further configured for, subtracting the corresponding gas resistance pressure value from the output pressure value of the apparatus body, and obtaining the actual pressure value.

In the embodiment of the present disclosure, when the patient is breathing, it will produce changes in airflow, therefore it will cause a pressure drop between the signal collection point and the end of the patient interface. The flow of the gas passing through the respiratory pipe 20 and the pressure drop between ΔP between the first end of the respiratory pipe and the end of the patient interface have a functional relationship ΔP=k*Flowⁿ, wherein n is slightly less than 2. The pressures P₁ of the first end of the respiratory pipe and the pressure P₂ of the end of the patient interface have a relationship of P₁=ΔP+P₂. Specifically, k and n may be constants, and the values of k and n may be measured by experiments on the circuit.

Specifically, the experiments on the circuit may include: operating the apparatus body 10, and placing the patient interface 30 in the air, at this time, the actual pressure P₂ at the patient interface 30 is 0. According to the formula P₁=ΔP+P₂, ΔP=P₁ may be obtained. Recording the flow value and the pressure drop value, multiple experiments, obtaining a correspondence relationship diagram of the flow and the pressure drop. According to the correspondence relationship diagram, the value of k and n may be obtained, that is, the correspondence relationship between the air resistance pressure value and the output flow value is obtained. Among them, the correspondence relationship between the air resistance pressure value and the output flow value may be a non-linear correspondence relationship.

Preferably, when the ventilation therapy apparatus is officially working, the patient interface 30 is inserted into the patient's nasal cavity, and the air resistance pressure acquisition unit 10313 detects the working pressure value outputted by the apparatus body 10 itself to the first end 201 of the respiratory pipe, and according to the air resistance characteristic corresponding to the working pressure value, and the corresponding air resistance pressure value is obtained. Specifically, according to the flow and the pressure drop diagram, the air resistance pressure value ΔP corresponding to the working pressure value in the flow and the pressure drop diagram may be determined, and according to the formula P₁=ΔP+P₂, the value of the airflow pressure P₂ at the patient interface may be obtained, the value of P₂ is the second pressure minus the air resistance pressure ΔP.

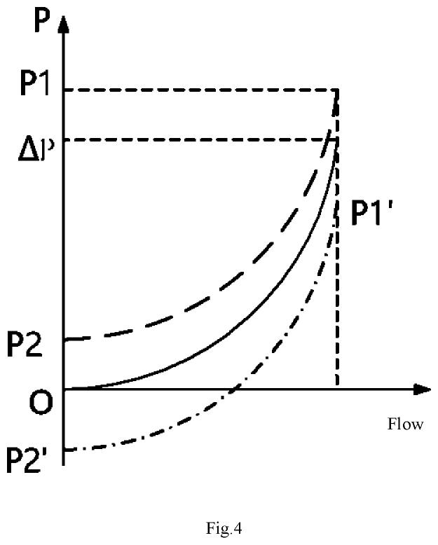

Specifically, referring to FIG. 4, it is shown a schematic diagram showing a flow-pressure drop of the patient's respiratory process according to the present disclosure. When the air supply system of the ventilation therapy apparatus is officially working, the patient interface 30 is inserted into the patient's nasal cavity, and the control module detects the airflow pressure P₁ and the flow F₀ outputted by the ventilation therapy apparatus itself to the first end of the respiratory pipe, the flow-pressure drop diagram may be used to express the gas resistance characteristics. According to the flow-pressure drop diagram, the theoretical output flow Fₜ may be calculated from P₁, or the pressure drop ΔP of the air flow through the circuit may be found from F₀, and according to the formula P₁=ΔP+P₂, the value of the airflow pressure P₂ at the patient interface may be obtained.

The air resistance characteristics include an air resistance characteristic of the respiratory pipe and an air resistance characteristic of the patient interface; the air resistance characteristic of the respiratory pipe is related to the cross-sectional area of the respiratory pipe, and the air resistance characteristic of the patient interface is related to the air outlet gap. Specifically, the patient interface fixing belt is worn on the patient's face, even after being worn correctly, the gas discharge path and the area of the gas path may vary with the physiological characteristics of the patient, and may vary with the strength of the patient when wearing it.

In an experiment, the pressure and flow rate tests are carried out through three types of nasal oxygen tubes of L, M and S, the specific experimental results may be referred to Table 1, so as to understand the pressure and flow characteristics of different types of nasal oxygen tubes at work.

TABLE 1

| L | | M | | S | |
|---|---|---|---|---|---|
| Flow | Pressure | Flow | Pressure | Flow | Pressure |
| (0.1 L/min) | (pa) | (0.1 L/min) | (pa) | (0.1 L/min) | (pa) |
| / | / | / | / | 100 | 72 |
| 150 | 64.75 | 150 | 101.5 | 150 | 145.25 |
| 200 | 109 | 199.95 | 169.875 | 200.025 | 249 |
| 250 | 152.4 | 250.075 | 244.05 | 249.975 | 362.75 |
| 300 | 204.25 | 300 | 329.5 | 300.075 | 493.25 |

Optionally, the signal collection point is disposed at the gas outlet of the apparatus body 10. In the embodiment of the present disclosure, the signal collection point of the apparatus body 10 may be the gas outlet of the apparatus body 10 or a position close to the gas outlet, and preferably, is configured to obtain the actual input pressure P₁ or flow rate F₀ of the first end 201 of the respiratory pipe 20.

In the embodiment of the present disclosure, when the patient is inhaling, the total flow outputted by the ventilation therapy apparatus is larger than the total flow by the ventilation therapy apparatus when the patient is exhaling. If the actual pressure value is less than the target pressure value, then it is determined that the patient is inhaling, meanwhile rising the output flow of the apparatus body to assist the patient to inhale; if the actual pressure value is larger than the target pressure value, then it is determined that the patient is exhaling, and reducing the output flow of the apparatus body to prevent the patient's exhaled gas from flowing back to the ventilation therapy apparatus. If the actual pressure value is equal to the target pressure value, then maintain the current rotating speed of the fan unchanged.

In addition, in another implementation, the size relationship of the patient's respiratory flow and the output flow of the ventilation therapy apparatus may be obtained from F=Fₜ−F₀ (Fₜ is the theoretical output flow calculated according to P₁, and F₀ is the second flow value of the signal collection point when the patient interface is inserted into the patient's nasal cavity), if F>0, it means that the flow rate output by the ventilation therapy apparatus is larger than the patient's respiratory flow. At this time, the patient is in the state of exhalation, or in the state of inhalation, and the inhalation volume is all provided by the ventilation therapy apparatus; if F<0, it means that the output flow of the ventilation therapy apparatus is less than the patient's inspiratory flow, and the patient will inhale some air from the environment. At this time, the patient's inhaled oxygen concentration cannot reach the disposed value. Therefore, if F>0 may be maintained during the operation of the ventilation therapy apparatus, and the relationship between the oxygen flow Fₒ₂ and the total flow F₀ or the air flow Fₐᵢᵣ is maintained in the relationship of the above equation, it may be ensured that the patient inhales the fixed oxygen concentration gas provided by the ventilation therapy apparatus.

In practical applications, when the patient wears the patient interface, because of its own air resistance, the patient interface will also have a certain pressure due to the patient's air resistance when not breathing. When the patient interface is removed, the patient interface is directly connected to the environment, the actual pressure P₂ is close to 0. Because the airway is negative pressure when inhaling, if the inhalation is strong, the pressure P₂ at the patient interface may drop to 0 or a negative value, but it will not be maintained for a long time, the pressure P₂ at the patient interface is determined to be close to 0 for a long time, it may be regarded as a non-use state.

Optionally, the ventilation therapy apparatus further comprises a second control module, when the patient interface is worn on the patient's nasal cavity, the second control module is configured for adjusting the output pressure value of the apparatus body to the target pressure value; and when the patient interface is not worn on the patient's nasal cavity, the second control module is configured for adjusting the output pressure value of the apparatus body to a preset pressure value, or the second control module is configured for controlling the apparatus body to stop running.

Therefore, in the embodiment of the present disclosure, when the pressure P₂ at the patient interface is close to 0, it is in the standby state, when the airflow pressure at the patient interface is close to 0 and maintained for the preset time, it means that the patient interface is not worn on the patient's nasal cavity, the patient does not use the air supply system of the ventilation therapy apparatus, and it is the standby state. When the patient interface is exposed to the air, at this time, the ventilation therapy apparatus may continue to output smaller output flow, to ensure the temperature and the humidity inside the respiratory pipe are constant, or directly control the apparatus body to stop running, to save power.

In addition, when the patient interface is worn on the patient's nasal cavity, the second control module is configured for adjusting the output pressure value of the apparatus body to the target pressure value, therefore the patient may receive oxygen supply quickly.

Optionally, the target pressure value comprises a target pressure value of an inspiratory phase and a target pressure value of an exhalation phase; the ventilation therapy apparatus includes a determination module, the determination module is configured for determining a respiratory phase according to the output pressure value and the output flow value acquired by the signal acquisition module, the respiratory phase includes the inspiratory phase and the exhalation phase;

when the determination module determines that the current is the inspiratory phase, the first control module adjusts the output flow of the apparatus body according to the actual pressure value and the target pressure value of the inspiratory phase; and when the determination module determines that the current is the exhalation phase, the first control module adjusts the output flow of the apparatus body according to the actual pressure value and the target pressure value of the exhalation phase.

In the embodiment of the present disclosure, according to the schematic diagram showing the flow-time of the patient's respiratory process shown in FIG. 2, we could know that, in the process of the patient using the ventilation therapy apparatus to breathe, there are two process: an inhalation phase and an exhalation phase. The determination module is configured to determine the respiratory phase according to the output pressure value and the output flow value obtained by the signal acquisition module. Therefore, for the ordinary ventilation therapy apparatus, in order to optimize the patient's breathing experience, it may provide the patient with two different levels of positive pressure during the patient's exhalation and inhalation. For the high-flow oxygen therapy apparatus, it may provide a larger flow when the patient inhales, therefore the patient may inhale more gas, and provide a smaller flow when exhaling, so as to avoid blockage of the patient's airway.

Specifically, before the patient uses the ventilation therapy apparatus, he will preset a target pressure value Pₜ according to his own situation, and the target pressure value Pₜ is the pressure at the patient's interface in an ideal state. According to the airflow pressure P₁ and the airflow flow F₀ output by the apparatus body 10, the value of the actual pressure P₂ at the patient interface may be calculated. Through real-time monitoring of P₂, and comparing P₂ with the target pressure value Pₜ, it may be determined which preset state the apparatus body is in, that is the exhalation state or the inhalation state. When the actual pressure value P₂ is larger than the target pressure value Pₜ, it is determined as the exhalation phase; if the actual pressure value P₂ is less than the target pressure value Pₜ, it is determined as the inhalation phase.

When it is determined that the patient is inhaling, the ventilation therapy apparatus, under the control of the first control module, makes the motor drive the fan to work at a relatively large rotating speed, therefore the ventilation therapy apparatus outputs a flow that is slightly larger than the patient's inhalation volume, until the pressure value at the patient interface reaches the target pressure value of the inhalation phase, to perform auxiliary inhalation.

When it is determined that the patient is exhaling, the ventilation therapy apparatus, under the control of the first control module, makes the motor drive the fan to work at a lower rotating speed, therefore the ventilation therapy apparatus outputs a smaller flow rate, until the pressure value at the patient interface reaches the target pressure value of the exhalation stage, to prevent the patient's exhaled gas from flowing back to the ventilation therapy apparatus.

Optionally, the apparatus body further comprises a positive pressure gas source and a humidifier, the positive pressure gas source is configured for providing an output gas, and the humidifier is configured for heating and humidifying the output gas, the humidifier is connected to an output end of the positive pressure gas source.

In practical applications, the gas people breathe has a certain amount of moisture, and the breathed gas has the highest breathing comfort at a certain temperature. Therefore, the gas provided by the positive pressure gas source may be heated and humidified through the humidifier, therefore it may meet the user's breathing needs and improve the breathing effect.

Optionally, the positive pressure gas source comprises a gas source body capable of outputting gas with a preset flow, and/or a centrifugal fan configured for pressurize air, the maximum rotation speed of the centrifugal fan is larger than or equal to 20000 r/min.

In the embodiment of the present disclosure, the positive pressure gas source may be a gas cylinder that stores a quantitative amount of breathing gas. In addition, the positive pressure gas source may also be outside air, and the ventilation therapy apparatus may transmit the gas provided by the positive pressure gas source through the centrifugal fan.

Optionally, the respiratory pipe further comprises a heating element configured for heating gas passing through the respiratory pipe, the rated power of the heating element is larger than 20 watts.

In the embodiment of the present disclosure, according to the different usage scenarios of the ventilation therapy apparatus, the temperature of the output gas is easily affected by the colder environment. In this case, the heating element configured to heat the gas passing through the respiratory pipe may be installed in the respiratory pipe. Preferably, the temperature of the output gas is heated in a cold environment, to improve the breathing experience of the patient.

Optionally, the respiratory pipe further comprises a temperature sensor, configured for monitoring the temperature of the gas passing through the respiratory pipe. The temperature sensor may real-time monitor the temperature of the gas in the respiratory pipe, therefore the ventilation therapy apparatus according to the monitored temperature, carries out the operation of correspondingly controlling the heating element to heat gas, and stops heating at the same time when the temperature is too high.

Optionally, the respiratory pipe and the apparatus body are connected through a gas path and a circuit, and the circuit and the gas path are on and off simultaneously.

In the embodiment of the present disclosure, the respiratory pipe and the apparatus body are connected through the gas path, which may output the gas provided by the apparatus body to the patient. In addition, the respiratory pipe and the apparatus body are connected through the circuit, and the electrical device in the respiratory pipe may also be electrically connected to the apparatus body, to realize the corresponding function of the electrical device. For example, the electrical device may include a humidifier, heating elements and temperature sensors, these devices need to be powered by the apparatus body, need to receive control signals transmitted by the apparatus body, and at the same time need to transmit corresponding status signals to the apparatus body.

In summary, the ventilation therapy apparatus according to the embodiment of the present disclosure, includes: an apparatus body, a respiratory pipe and a patient interface. The apparatus body further includes: a signal acquisition module, a target pressure acquisition module and a first control module. The signal acquisition module is configured for acquiring the output pressure value and the output flow value of the signal collection point of the apparatus body; the target pressure acquisition module is configured for acquiring the target pressure value at the patient interface; the first calculation module is configured for calculating the actual pressure value at the patient interface according to the output pressure value and the output flow value of the signal collection point; the first control module is configured for adjusting the output flow of the apparatus body according to the actual pressure value and the target pressure value. In the present disclosure, it is capable to determine the actual pressure value at the patient interface by the output parameters feedback of the signal collection point of the apparatus body, determine the patient's respiratory state according to the comparison between the actual pressure value and the target pressure value, and output the gas with a corresponding threshold, therefore the gas pressure of the airflow received by the patient may reach a preset target pressure range, achieve the therapeutic effect, and ensure the patient inhales the gas with a fixed oxygen concentration provided by the ventilation therapy apparatus. In addition, in the present disclosure, it may dispose the heating element inside the respiratory pipe, the heating element is configured for heating the gas passing through the respiratory pipe, preferably, the temperature of the output gas is heated in a cold environment, to improve the breathing experience of the patient. The gas may also be heated and humidified by the humidifier, therefore it may meet the user's breathing needs and improve the breathing effect

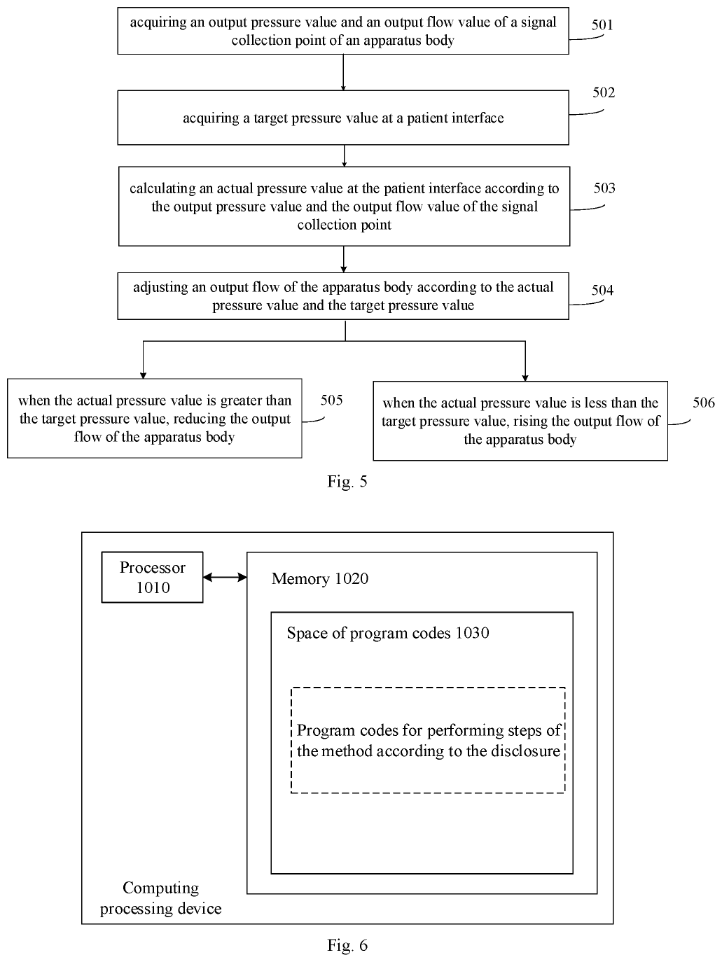

Referring to FIG. 5, it is shown a flow chart showing steps of the method for controlling the ventilation therapy apparatus according to the present disclosure, the method for controlling the ventilation therapy apparatus, the ventilation therapy apparatus constructs a semi-open gas path, wherein the method comprises:

Step 501, acquiring an output pressure value and an output flow value of a signal collection point of an apparatus body.

Specifically, in this step, the output pressure value P₁ and the output flow value F₀ of the signal collection point of the apparatus body may be obtained. Wherein, the signal collection point of the apparatus body may be the gas outlet of the apparatus body or a position close to the gas outlet, and preferably, is configured to obtain the actual input pressure P₁ or flow rate F₀ of the first end of the respiratory pipe.

Step 502, acquiring a target pressure value at a patient interface.

In the embodiment of the present disclosure, before the patient uses the ventilation therapy apparatus, he will preset a target pressure value Pₜ according to his own situation, and the target pressure value Pₜ is the pressure at the patient's interface in an ideal state. According to the airflow pressure P₁ and the airflow flow F₀ output by the apparatus body, the value of the actual pressure P₂ at the patient interface may be calculated. Through real-time monitoring of P₂, and comparing P₂ with the target pressure value Pₜ, it may be determined which preset state the apparatus body is in, that is the exhalation state or the inhalation state, and according to the current state of the apparatus body, the fan of the apparatus body 10 is controlled to run at the corresponding preset rotating speed, and outputs the airflow corresponding to the preset threshold, for example, when it is determined that the patient is inhaling, the air supply system of the ventilation therapy apparatus outputs a flow that is slightly larger than the patient's inhalation volume for auxiliary inhalation; when the patient is determined to exhale, the air supply system of the ventilation therapy apparatus outputs a smaller flow rate, to prevent the patient's exhaled gas from flowing back to the ventilation therapy apparatus.

It should be noted that, the target pressure value Pₜ needs to be a positive pressure value, that is, the target pressure value Pₜ is larger than the atmospheric pressure value. Because in the embodiment of the present disclosure, the ventilation therapy apparatus uses an open gas path, it is necessary to ensure that the positive pressure is maintained in the nasal cavity, to ensure that the human body will not directly inhale outside air.

Step 503, calculating an actual pressure value at the patient interface according to the output pressure value and the output flow value of the signal collection point.

In this step, either the theoretical output flow rate Fₜ may be calculated from P₁, or the pressure drop ΔP of the air flow through the tube may be calculated from F₀, and according to the formula P₁=ΔP+P₂, the value of the airflow pressure P₂ at the patient interface may be obtained.

Optionally, the step 503 further comprises:

Sub-step S031, acquiring a gas resistance pressure value from the signal collection point to the patient interface.

For details of this step, reference may be made to the above description of the air resistance pressure acquisition module 1031, which is not repeated here.

Sub-step S032, subtracting the gas resistance pressure value from the output pressure value, and obtaining the actual pressure value.

For details of this step, reference may be made to the above description of the second calculation module 1032, which is not repeated here.

Optionally, the step 503 further comprises:

Sub-step S033, acquiring the output pressure value and the output flow value of the signal collection point of the apparatus body;

For details of this step, reference may be made to the above description of the flow acquisition module 10311, which is not repeated here.

Sub-step S034, acquiring, under different pressure states, corresponding test flow values when the patient interface is vacant, and acquiring a gas resistance characteristic from the signal collection point to the patient interface, the gas resistance characteristic includes a correspondence relationship between the output pressure value and the output flow value.

For details of this step, reference may be made to the above description of the gas resistance characteristic acquisition module 10312, which is not repeated here.

Sub-step S035, according to the output flow value of the apparatus body in working state and the corresponding gas resistance characteristic, acquiring the corresponding gas resistance pressure value.

For details of this step, reference may be made to the above description of the gas resistance pressure acquisition unit 10313, which is not repeated here.

Sub-step S036, subtracting the corresponding gas resistance pressure value from the output pressure value of the apparatus body, and obtaining the actual pressure value.

For details of this step, reference may be made to the above description of the second calculation module 1032, which is not repeated here.

Step 504, adjusting an output flow of the apparatus body according to the actual pressure value and the target pressure value.

Step 505, when the actual pressure value is larger than the target pressure value, reducing the output flow of the apparatus body.

In the embodiment of the present disclosure, before the patient uses the air supply system of the ventilation therapy apparatus, the target pressure value Pₜ will be preset according to their own conditions, and the target pressure value Pₜ is the pressure value in an ideal state free from the interference of the pressure drop. When the patient uses the ventilation therapy apparatus, due to the interference of the pressure drop, which will cause the actual pressure P₂ at the patient interface is different from the target pressure value Pₜ, and the influence of this pressure drop on the actual pressure P₂ at the patient interface may be determined by the pressure compensation module based on the comparison result between P₂ and Pₜ. If the pressure compensation module determines that the actual pressure P₂ at the patient interface is larger than the target pressure value Pₜ, it is determined that the current working state of the apparatus body 10 is the exhalation state, meanwhile a control instruction is sent to the motor by the pressure compensation module, therefore the motor drives the fan to work at a lower rotating speed, and output a smaller first output threshold airflow, to prevent the patient's exhaled gas from flowing back to the ventilation therapy apparatus, until the actual pressure value is equal to the target pressure value.

Step 506, when the actual pressure value is less than the target pressure value, rising the output flow of the apparatus body.

In this step, if the pressure compensation module determines that the actual pressure P₂ at the patient interface is less than the target pressure value Pₜ, then it is determined that the current working state of the apparatus body is the inhalation state. At this time, a control instruction is sent to the motor by the pressure compensation module, therefore the motor drives the fan to work at a relatively high rotating speed, and output a larger second output threshold airflow, to perform auxiliary inhalation.

Optionally, in an implementation manner, it may also include:

Step A1, when the patient interface is worn on the patient's nasal cavity, adjusting the output pressure value of the apparatus body to the target pressure value; and

Step A2, when the patient interface is not worn on the patient's nasal cavity, adjusting the output pressure value of the apparatus body to a preset pressure value which is less than the target pressure value, or controlling the apparatus body to stop running.

In the embodiment of the present disclosure, when the pressure P₂ at the patient interface is close to 0, it is in the standby state, when the airflow pressure at the patient interface is close to 0 and maintained for the preset time, it means that the patient interface is not worn on the patient's nasal cavity, the patient does not use the air supply system of the ventilation therapy apparatus, and it is the standby state. When the patient interface is exposed to the air, at this time, the ventilation therapy apparatus may continue to output smaller output flow, to ensure the temperature and the humidity inside the respiratory pipe are constant, or directly control the apparatus body to stop running, to save power.

In addition, when the patient interface is worn on the patient's nasal cavity, the second control module is configured for automatically adjusting the output pressure value of the apparatus body to the target pressure value, therefore the patient may receive oxygen supply quickly.

Optionally, in another implementation manner, it may also include:

Step B1, determining a respiratory phase according to the output pressure value and the output flow value, the respiratory phase includes the inspiratory phase and the exhalation phase.

Step B2, acquiring a target pressure value of the inspiratory phase and a target pressure value of the exhalation phase at the patient interface.

Specifically, the patient may preset the target pressure value of the inspiration phase and the target pressure value of the expiration phase, and the target pressure value of the inspiration phase and the target pressure value of the expiration phase are the pressures of the patient interface during inhalation and exhalation in an ideal state.

Step B3, if it is determined that the he current is inspiratory phase, adjusting the output flow of the apparatus body according to the actual pressure value and the target pressure value of the inspiratory phase.

When it is determined that the patient is inhaling, the ventilation therapy apparatus, under the control of the first control module, makes the motor drive the fan to work at a relatively large rotating speed, therefore the ventilation therapy apparatus outputs a flow that is slightly larger than the patient's inhalation volume, until the pressure value at the patient interface reaches the target pressure value of the inhalation phase, to perform auxiliary inhalation.

Step B4, if it is determined that the he current is exhalation phase, adjusting the output flow of the apparatus body according to the actual pressure value and the target pressure value of the exhalation phase.

When it is determined that the patient is exhaling, the ventilation therapy apparatus, under the control of the first control module, makes the motor drive the fan to work at a lower rotating speed, therefore the ventilation therapy apparatus outputs a smaller flow rate, until the pressure value at the patient interface reaches the target pressure value of the exhalation stage, to prevent the patient's exhaled gas from flowing back to the ventilation therapy apparatus.

In summary, the method for controlling the ventilation therapy apparatus according to the embodiment of the present disclosure, includes: acquiring an output pressure value and an output flow value of a signal collection point of an apparatus body; acquiring a target pressure value at a patient interface; calculating an actual pressure value at a patient interface according to the output pressure value and the output flow value of the signal collection point; adjusting the output flow of the apparatus body according to the actual pressure value and the target pressure value; when the actual pressure value is larger than the target pressure value, reducing the output flow of the apparatus body; and when the actual pressure value is less than the target pressure value, rising the output flow of the apparatus body. In the present disclosure, it is capable to determine the actual pressure value at the patient interface by the output parameters feedback of the signal collection point of the apparatus body, determine the patient's respiratory state according to the comparison between the actual pressure value and the target pressure value, and output the gas with a corresponding threshold, therefore the gas pressure of the airflow received by the patient may reach a preset target pressure range, and achieve the therapeutic effect.

The various component embodiments of the present disclosure may be implemented by hardware, or by software modules running on one or more processors, or by a combination of them. Those skilled in the art should understand that, a microprocessor or a digital signal processor (DSP) may be used in practice to implement some or all of the functions of some or all of the components in the computing processing device according to the embodiments of the present disclosure. The present disclosure may also be implemented as a device or device program (for example, a computer program and a computer program product) for executing part or all of the methods described herein. Such a program for implementing the present application may be stored on a computer-readable medium, or may have the form of one or more signals. Such a signal may be downloaded from an Internet website, or provided on a carrier signal, or provided in any other form.

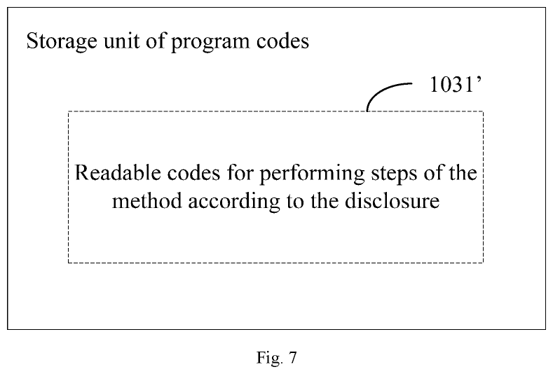

For example, FIG. 6 is a computing processing device that may implement the method according to the present disclosure provided in an embodiment of the present disclosure. The computing processing device traditionally includes a processor 1010 and a computer program product in the form of a memory 1020 or a computer readable medium. The memory 1020 may be an electronic memory such as flash memory, EEPROM (Electrically Erasable Programmable Read Only Memory), EPROM, hard disk, or ROM. The memory 1020 has a storage space 1030 for executing the program code 1031 of any method step in the above method. For example, the storage space 1030 for program codes may include various program codes 1031 respectively used to implement various steps in the above method. These program codes may be read from or written into one or more computer program products. These computer program products include program code carriers such as hard disks, compact disks (CDs), memory cards, or floppy disks. Such computer program products are usually portable or fixed storage modules as described with reference to FIG. 7. The storage module may have storage segments, storage spaces, etc., arranged similarly to the memory 1020 in the computing processing device of FIG. 6. The program code may be compressed in an appropriate form, for example. Generally, the storage module includes computer-readable code 1031′, that is, code that may be read by a processor such as 1010, which, when run by the computing processing device, causes the computing processing device to execute the various steps of the method described above.

In this application, a computer-readable recording medium includes any mechanism for storing or transmitting information in a computer (for example, a computer) readable form. For example, machine-readable medias include read-only memory (ROM), random access memory (RAM), magnetic disk storage media, optical storage media, flash storage media, electrical, optical, acoustic, or other forms of propagated signals (for example, carrier waves, infrared signal, digital signal, etc.) etc.

The “one embodiment”, “an embodiment” or “one or more embodiments” referred to herein means that a specific feature, structure, or characteristic described in combination with the embodiment is included in at least one embodiment of the present disclosure. In addition, please note that the word examples “in one embodiment” here do not necessarily all refer to the same embodiment.

In the description provided here, a lot of specific details are explained. However, it may be understood that the embodiments of the present disclosure may be practiced without these specific details. In some instances, well-known methods, structures, and technologies are not shown in detail, so as not to obscure the understanding of this specification.

In the claims, any reference signs placed between parentheses should not be constructed as a limitation to the claims. The word “comprise” does not exclude the presence of elements or steps not listed in the claims. The word “a” or “an” preceding an element does not exclude the presence of multiple such elements. The disclosure may be realized by means of hardware including several different elements and by means of a suitably programmed computer. In the module claims enumerating several devices, several of these devices may be embodied in the same hardware item. The use of the words first, second, and third, etc. do not indicate any order. These words may be interpreted as names.

Finally, it should be noted that the above embodiments are only used to illustrate the technical solutions of the present disclosure, not to limit them; although the application has been described in detail with reference to the foregoing embodiments, those of ordinary skill in the art should understand that: it is still possible to modify the technical solutions described in the foregoing embodiments, or equivalently replace some of the technical features; and these modifications or replacements do not cause the essence of the corresponding technical solutions to deviate from the spirit and the scope of the technical solutions of the embodiments of the present disclosure.

1. A ventilation therapy apparatus, comprising:
an apparatus body, configured for outputting gas with a preset pressure and a preset flow, wherein the apparatus body includes a gas outlet;
a respiratory pipe, including a first end and a second end which communicates with each other, wherein the first end of the respiratory pipe communicates with the gas outlet of the apparatus body;
a patient interface, wherein the second end of the respiratory pipe is connected to the patient interface, the patient interface is configured for being worn on a patient's nasal cavity, when the patient interface is worn on the patient's nasal cavity, a gas outlet gap is disposed between the patient interface and the patient's nasal cavity;
wherein the apparatus body is configured for performing operations comprising:
acquiring an output pressure value and an output flow value of a signal collection point of the apparatus body;
acquiring a target pressure value at the patient interface;
calculating an actual pressure value at the patient interface according to the output pressure value and the output flow value of the signal collection point;
adjusting an output flow of the apparatus body according to the actual pressure value and the target pressure value;
when the actual pressure value is larger than the target pressure value, reducing the output flow of the apparatus body; and
when the actual pressure value is less than the target pressure value, rising the output flow of the apparatus body.

2. The ventilation therapy apparatus according to claim 1, wherein the operation of calculating an actual pressure value at the patient interface according to the output pressure value and the output flow value of the signal collection point comprises:
acquiring a gas resistance pressure value from the signal collection point to the patient interface; and
subtracting the gas resistance pressure value from the output pressure value, and obtaining the actual pressure value.

3. The ventilation therapy apparatus according to claim 2, wherein the operation of acquiring a gas resistance pressure value from the signal collection point to the patient interface comprises:
acquiring, under different pressure states, corresponding test flow values when the patient interface is vacant, and acquiring a gas resistance characteristic from the signal collection point to the patient interface, wherein the gas resistance characteristic includes a correspondence relationship between the gas resistance pressure value and the output flow value;
acquiring the corresponding gas resistance pressure value according to the output flow value of the apparatus body in working state and the corresponding gas resistance characteristic;
subtracting the corresponding gas resistance pressure value from the output pressure value of the apparatus body, and obtaining the actual pressure value.

4. The ventilation therapy apparatus according to claim 1, wherein the signal collection point is disposed at the gas outlet of the apparatus body.

5. The ventilation therapy apparatus according to claim 1, wherein the target pressure value comprises a target pressure value of an inspiratory phase and a target pressure value of an exhalation phase;
the apparatus body of the ventilation therapy apparatus is further configured for performing operations comprising:
determining a respiratory phase according to the acquired output pressure value and the output flow value, the respiratory phase includes the inspiratory phase and the exhalation phase;
when it is determined that the current is the inspiratory phase, adjusting the output flow of the apparatus body according to the actual pressure value and the target pressure value of the inspiratory phase; and
when it is determined that the current is the exhalation phase, adjusting the output flow of the apparatus body according to the actual pressure value and the target pressure value of the exhalation phase.

6. The ventilation therapy apparatus according to claim 1, wherein the apparatus body further comprises a positive pressure gas source and a humidifier, the positive pressure gas source is configured for providing an output gas, and the humidifier is configured for heating and humidifying the output gas, wherein the humidifier is connected to an output end of the positive pressure gas source.

7. The ventilation therapy apparatus according to claim 6, wherein the positive pressure gas source comprises a gas source body capable of outputting gas with a preset flow, and/or a centrifugal fan configured for pressurize air, wherein the maximum rotation speed of the centrifugal fan is larger than or equal to 20000 r/min.

8. The ventilation therapy apparatus according to claim 1, wherein the respiratory pipe further comprises a heating element configured for heating gas passing through the respiratory pipe, the rated power of the heating element is larger than 20 watts.

9. The ventilation therapy apparatus according to claim 8, wherein the respiratory pipe further comprises a temperature sensor, configured for monitoring the temperature of the gas passing through the respiratory pipe.

10. The ventilation therapy apparatus according to claim 1, wherein the respiratory pipe and the apparatus body are connected through a gas path and a circuit, and the circuit and the gas path are on and off simultaneously.

11. The ventilation therapy apparatus according to claim 1, wherein the apparatus body of the ventilation therapy apparatus is further configured for:
when the patient interface is worn on the patient's nasal cavity, adjusting the output pressure value of the apparatus body to the target pressure value; and
when the patient interface is not worn on the patient's nasal cavity, adjusting the output pressure value of the apparatus body to a preset pressure value, or the second control module is configured for controlling the apparatus body to stop running.

12. A method for controlling a ventilation therapy apparatus, wherein the ventilation therapy apparatus constructs a semi-open gas path, the method comprises:
acquiring an output pressure value and an output flow value of a signal collection point of an apparatus body;
acquiring a target pressure value at a patient interface;
calculating an actual pressure value at the patient interface according to the output pressure value and the output flow value of the signal collection point;
adjusting an output flow of the apparatus body according to the actual pressure value and the target pressure value;
when the actual pressure value is larger than the target pressure value, reducing the output flow of the apparatus body; and
when the actual pressure value is less than the target pressure value, rising the output flow of the apparatus body.

13. The method according to claim 12, wherein the step of calculating an actual pressure value at the patient interface according to the output pressure value and the output flow value of the signal collection point, comprises:
acquiring a gas resistance pressure value from the signal collection point to the patient interface; and
subtracting the gas resistance pressure value from the output pressure value, and obtaining the actual pressure value.

14. The method according to claim 12, wherein the step of calculating an actual pressure value at the patient interface according to the output pressure value and the output flow value of the signal collection point, comprises:
acquiring, under different pressure states, corresponding test flow values when the patient interface is vacant, and acquiring a gas resistance characteristic from the signal collection point to the patient interface, wherein the gas resistance characteristic includes a correspondence relationship between the gas resistance pressure value and the output flow value;
acquiring the corresponding gas resistance pressure value according to the output flow value of the apparatus body in working state and the corresponding gas resistance characteristic; and
subtracting the corresponding gas resistance pressure value from the output pressure value of the apparatus body, and obtaining the actual pressure value.

15. The method according to claim 12, wherein the method further comprises:
when the patient interface is worn on the patient's nasal cavity, adjusting the output pressure value of the apparatus body to the target pressure value; and
when the patient interface is not worn on the patient's nasal cavity, adjusting the output pressure value of the apparatus body to a preset pressure value which is less than the target pressure value, or controlling the apparatus body to stop running.

16. The method according to claim 12, wherein after acquiring an output pressure value and an output flow value of a signal collection point of an apparatus body, the method further comprises:
determining a respiratory phase according to the output pressure value and the output flow value, wherein the respiratory phase includes the inspiratory phase and the exhalation phase;
acquiring a target pressure value of the inspiratory phase and a target pressure value of the exhalation phase at the patient interface;
if it is determined that the he current is inspiratory phase, adjusting the output flow of the apparatus body according to the actual pressure value and the target pressure value of the inspiratory phase; and
if it is determined that the he current is exhalation phase, adjusting the output flow of the apparatus body according to the actual pressure value and the target pressure value of the exhalation phase.

17. (canceled)

18. A non-transitory computer readable medium, storing computer program, when the computer program is executed by one or more processors of a computing device, the computing device performs operations comprising:
acquiring an output pressure value and an output flow value of a signal collection point of an apparatus body;
acquiring a target pressure value at a patient interface;
calculating an actual pressure value at the patient interface according to the output pressure value and the output flow value of the signal collection point;
adjusting an output flow of the apparatus body according to the actual pressure value and the target pressure value;
when the actual pressure value is larger than the target pressure value, reducing the output flow of the apparatus body; and
when the actual pressure value is less than the target pressure value, rising the output flow of the apparatus body.

19. The non-transitory computer readable medium according to claim 18, wherein the operation of calculating an actual pressure value at the patient interface according to the output pressure value and the output flow value of the signal collection point, comprises:
acquiring a gas resistance pressure value from the signal collection point to the patient interface; and
subtracting the gas resistance pressure value from the output pressure value, and obtaining the actual pressure value.

20. The non-transitory computer readable medium according to claim 18, wherein the operation of calculating an actual pressure value at the patient interface according to the output pressure value and the output flow value of the signal collection point, comprises:
acquiring, under different pressure states, corresponding test flow values when the patient interface is vacant, and acquiring a gas resistance characteristic from the signal collection point to the patient interface, wherein the gas resistance characteristic includes a correspondence relationship between the gas resistance pressure value and the output flow value;
acquiring the corresponding gas resistance pressure value according to the output flow value of the apparatus body in working state and the corresponding gas resistance characteristic; and
subtracting the corresponding gas resistance pressure value from the output pressure value of the apparatus body, and obtaining the actual pressure value.

21. The non-transitory computer readable medium according to claim 18, wherein the operation further comprises:
when the patient interface is worn on the patient's nasal cavity, adjusting the output pressure value of the apparatus body to the target pressure value; and
when the patient interface is not worn on the patient's nasal cavity, adjusting the output pressure value of the apparatus body to a preset pressure value which is less than the target pressure value, or controlling the apparatus body to stop running.
